(19) United States
(12) Patent Application Publication
(10) Pub. No.: US 2018/0303986 A1
(43) Pub. Date: Oct. 25, 2018

(54) Implanted Medical Driveline Strain Relief Device

(71) Applicant: G.B. Kirby Meacham, Shaker Heights, OH (US)

(72) Inventor: G.B. Kirby Meacham, Shaker Heights, OH (US)

(21) Appl. No.: 15/530,918

(22) PCT Filed: Nov. 16, 2015
(86) PCT No.: PCT/US2015/060815

§ 371 Date: Mar. 24, 2017

Related U.S. Application Data
(60) Provisional application No. 62/082,761, filed on Nov. 21, 2014.

(51) Int. Cl. A61M 1/10

(52) U.S. Cl. CPC A61M 1/1008; A61M 2025/0098; A61M 2205/0205; A61M 2205/0222

(57) ABSTRACT

The strain relief device of this invention reduces injury and danger of serious infection at driveline skin exit sites caused by routine patient activities and accidental stresses on the drivelines of ventricle assist devices and similar externally powered implanted medical devices. The device comprises a sleeve that forms an adherent biological interface with the skin and compliant seals between the sleeve and driveline. The compliant seals mechanically decouple linear and rotary driveline motions from the sleeve, while isolating the patients internal tissue from the outside environment. This mechanical decoupling reduces stresses and injury to the adherent interface, thereby reducing the risk of bacterial entry and infection. The device may be inserted into the patient as part of the driveline assembly implantation procedure, and is positioned such that the skin contacts and adheres to the outer diameter of the sleeve. It may also be partially or completely replaced or retrofitted to previously implanted drivelines in a relatively simple procedure.

### FIELD OF THE INVENTION

The present invention is related to cables or “drivelines” connecting an external power supply to a medical device such as a ventricular assist device (VAD) implanted within a patient's body. More generally, it is related to methods and devices for reducing injury and infection at an exit site where a cable or tube passes through the patient's skin.

### BACKGROUND OF THE INVENTION

Implanted VAD systems comprise an implanted pump that takes over at least part of a damaged heart's pumping function to improve the patient's ability to carry out the tasks of daily life. They may be used as temporary bridges to a heart transplant for periods of weeks to months or become permanent installations. In either case an electrical power cable or driveline is installed to connect to an external power supply and device controller. This driveline is tunneled through body tissue during the VAD implantation surgery, and exits through the skin. The driveline exit site in the skin is sealed by the natural tendency of living skin tissue to adhere to a compatible penetrating foreign object, and under favorable conditions forms a healthy adherent interface that prevents dangerous bacterial invasion and penetration along the driveline. The problem is that the adherent interface between the driveline and the skin is mechanically fragile. Routine patient activities as well as accidental events result in torsional, pull and bending loads on the driveline that apply stresses to the adherent interface that often cause tissue injury and open a path for bacterial invasion and infection along the driveline path through the patient's tissue. Such infections are a leading cause of complications, and may lead to major medical interventions or death. In some cases the driveline cable is covered in velour to improve mechanical bonding through tissue ingrowth, but the stresses are often significant enough that injury and infection still occur. There is therefore a clear need for a device or method to reduce tissue stress and injury at the driveline exit site resulting from routine activities such as patient movement and accidental events such as a strong tug on the driveline. Preferably, such a method or device could be applied as part of the original VAD implant procedure or to existing implanted systems, and is easily repairable in the event or damage.

### SUMMARY OF THE INVENTION

The present invention provides a compliant sealed connection between the driveline and the adherent interface with the skin that reduces adherent interface stresses during routine activities and accidental events that move the driveline relative to the patient's body. The device is essentially a short sliding sleeve surrounding the driveline cable that is preferably inserted into the patient as part of the driveline assembly, and is positioned such that the skin contacts and adheres to the outer diameter of the sleeve. All or part of the sleeve may have a velour surface to promote tissue adhesion. Axially and torsionally flexible elastomer bellows and seals at the inner and outer ends of the sleeve allows the driveline cable to move axially or rotate independently of the sleeve to minimize forces on the sleeve and the adherent tissue, while isolating the annulus between the sleeve and the cable from body fluids and external contaminants. Together the bellows form a double barrier between the external environment and the patient's subcutaneous tissue. Each elastomer bellows includes a ring that grips the driveline cable and forms an elastically loaded seal. The elastic seals are static seals in normal service, but may be slid manually to adjust the bellows position or slip under driveline force to relieve stress in an accidental event. It is expected that the inner bellows elastic seal will often be locked into position by biological encapsulation caused by the patient's foreign body reaction after a period of time. Biological encapsulation could increase the force on the sleeve in an accidental event, particularly a strong tug on the driveline, but it is likely that the sleeve will be well enough integrated into the tissue at this time that damage will be minimized.

The sleeve and the two bellows are intended to last the life of the driveline, but the outside bellows only or the entire device may be replaced if necessary. Replacement is possible since both parts are made of available implant-grade elastomers such as urethane or a urethane-silicone copolymer that have sufficient elasticity to be stretched and passed over the driveline connector. In most cases it is expected the device will be applied during the VAD implant surgical procedure, although the design includes provisions for device retrofit to existing implants without removing the VAD.

### BRIEF DESCRIPTION OF THE DRAWINGS

In the drawings, in which like reference numbers indicate corresponding parts throughout the several views,

FIG. 1 is a sectional view illustrating a typical driveline installation using the inventive device in a patient; and

FIG. 2 is an exploded perspective view illustrating the geometry of the inventive device components.

### DETAILED DESCRIPTION OF THE PREFERRED EMBODIMENTS

The following description and claims are in reference to implanted VAD system drivelines, but it is understood that the inventive device and methods are applicable to stress, injury and infection reduction for other implanted cables or tubes that exit through the skin. While the figures are schematic in that they show a linear geometry of the driveline and the device, in reality the driveline is flexible and will generally be curved. The device sleeve and bellows are formed from low modulus elastomers, and will therefore bend easily to follow the curvature of the driveline cable.

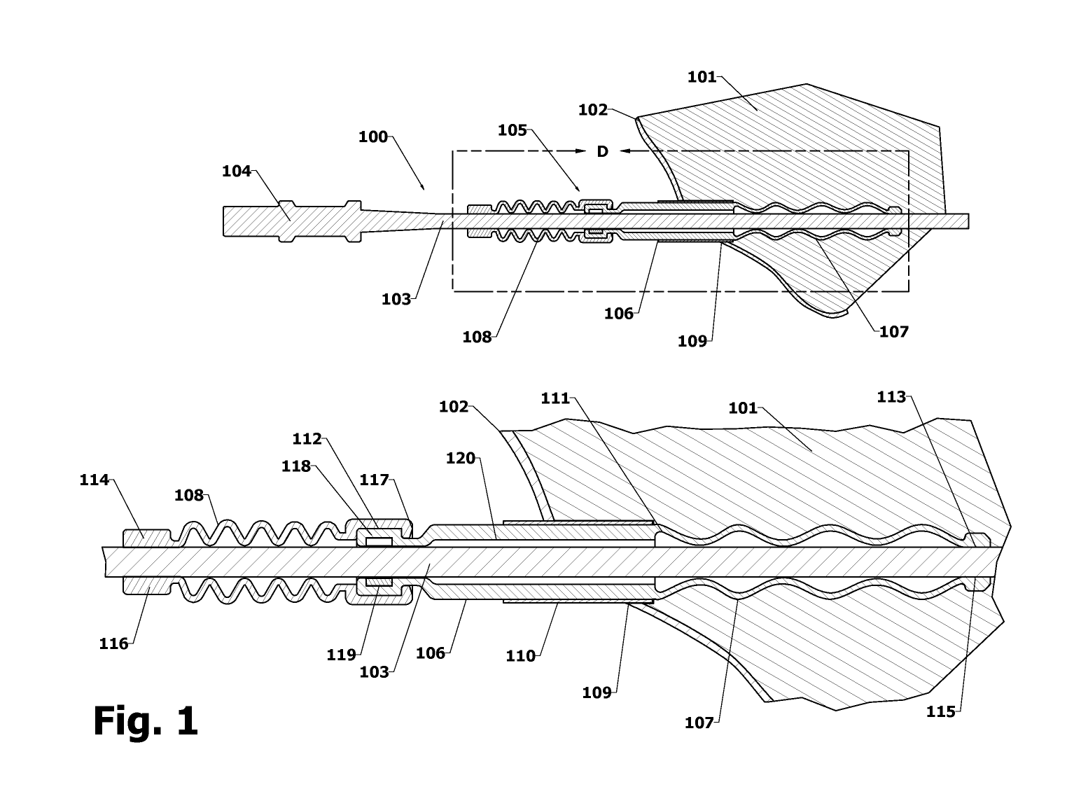

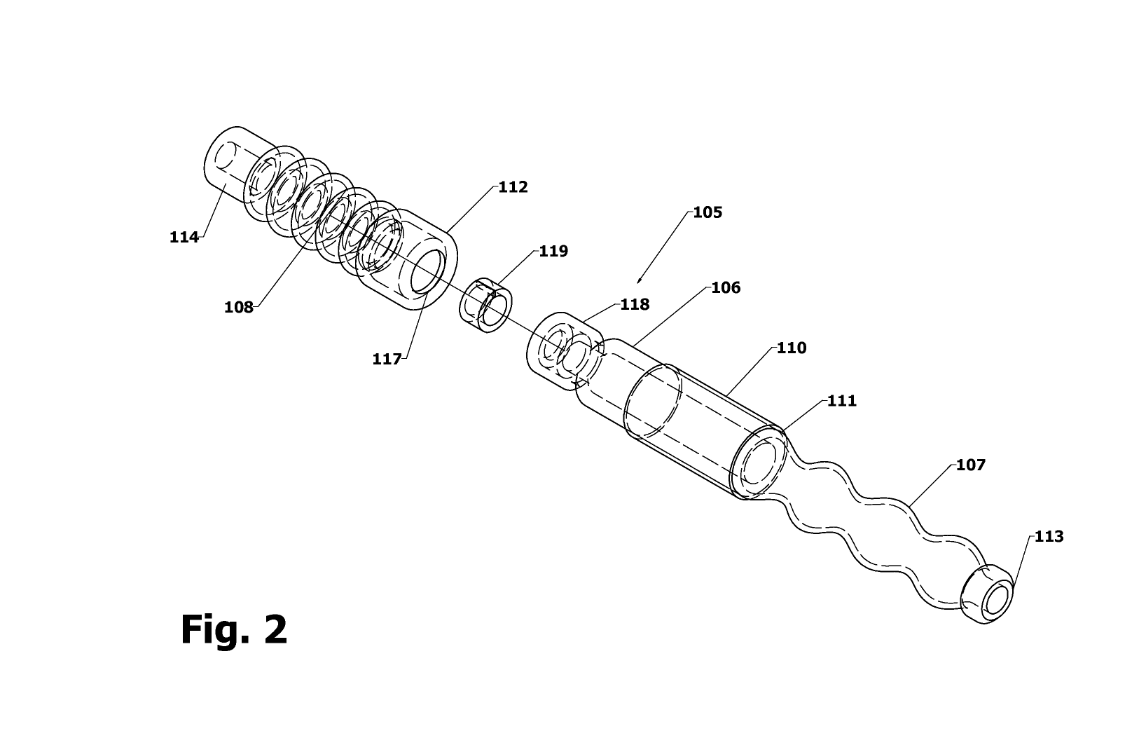

FIG. 1 illustrates the cross section of a preferred embodiment of the invention as applied to an implanted VAD driveline, and FIG. 2 shows the components comprising the inventive device. The driveline 100 powering the VAD (not shown) passes through a channel tunneled in the patient's subcutaneous tissue 101 and emerges from the skin 102. The driveline 100 comprises a cable 103 with a generally circular cross section and a larger diameter connector 104 that engages the external power supply and controller (not shown). The device assembly 105 comprises a sleeve 106, an inner bellows 107 and an outer bellows 108. The assembly 105 is coaxial with the cable 103, and positioned such that the skin 102 contacts the outer diameter of the sleeve 106. The skin 102 and a portion of the subcutaneous tissue 101 form an adherent interface 109 with the sleeve 106 in the process of healing after the implantation procedure. Optionally, at least a portion of the sleeve 106 is covered by velour 110 bonded to the sleeve to promote tissue adhesion. Cable 103 is a loose fit within sleeve 106 such that the cable is free to rotate or move axially within the sleeve without applying direct axial force or torque to the sleeve and the adherent joint 109. The inner bellows 107 and the outer bellows 108 bellows are formed of soft elastomer, and their large ends 111 and 112 are effectively part of the sleeve 106. Their small ends 113 and 114 elastically grip the cable 103 to form static seals 115 and 116. Static seals 115 and 116 may be slid to change position to adjust the initial bellows compression. Together bellows 107 and 108 form highly compliant seals between the sleeve 106 and the cable 103 that allow relative motion between the driveline cable 103 and the sleeve 106, while applying only indirect elastic axial force or torque to the sleeve and the adherent joint 109. In combination, bellows 107 and 108 form redundant seals separating the patient's subcutaneous tissue 101 from outside contaminants including pathogens while protecting the adherent joint from excessive stress and injury during normal activities.

The device also provides a measure of protection in extreme events such as a sharp accidental tug on the drive line assembly 100. The elastically loaded seals 115 and 116 are static seals in normal service, but may slip under driveline force to relieve stress in an extreme event. It is expected that after a period of time the inner bellows elastic seal may be locked into position on the driveline cable 103 by biological encapsulation caused by the patient's foreign body reaction. Biological encapsulation is discussed by Ratner in the Journal of Controlled Release 78 (2002) 211-218. Encapsulation could increase the force on the sleeve in an accidental event, particularly a strong tug on the driveline, but it is likely that the sleeve will be well enough integrated into the tissue at this time that injury will be minimized.

The preferred embodiment shown in FIG. 1 and FIG. 2 incorporates optional design features that permit device repair or replacement. The inner bellows 107 and the sleeve 106 are a single part and the outer bellows 108 is a separate part. The outer bellows 108 is stretched such that a lip 117 engages an attachment land 118 on the outer end of the sleeve, and forms a static seal with the land. A split support ring 119 is inserted in a groove 120 inside the land 118 to carry the inward radial force from the stretched bellows 108 to assure a good seal and prevent unwanted contact and friction between the sleeve 106 and the cable 103. The support ring 119 is a resilient polymer that can be spread to slip over the cable 103 from the side for installation or removal. The sleeve 106 and the bellows 107 and 108 are composed of a commercially available implant-grade elastomer such as urethane or a urethane-silicone copolymer that has sufficient elasticity to be stretched and passed over the driveline connector 104 to facilitate replacement. As an example DSM BioSpan segmented polyurethane has biocompatibility, strengths above 6000 psi, elongation over 900%, and is used in high elasticity applications including cardiac catheter balloons. If velour 110 is applied to the outside diameter of the sleeve 106, it preferably has a fabric structure such as knit that allows it to stretch with the sleeve during a replacement installation. The inventive device assembly 105 has an advantage beyond reducing axial force or torque applied to the adherent interface 109 as a result of driveline 100 motions. The enlarged perimeter of the adherent interface 109 with the sleeve 106, compared to a conventional adherent interface with the driveline cable 103, increases the interface area and is expected to increase its strength. While in theory a longer adherent interface perimeter increases the opportunity for infection, this is believed to be outweighed by a significant reduction in mechanical injury to the adherent interface tissue.

The device assembly 105 is preferably installed as part of the VAD implant procedure. The device is assembled on the driveline prior to implantation, preferably in the VAD system production setting, but it could be assembled in the operating room using sterile components. After tunneling through the subcutaneous tissue 101, the device assembly 105 is passed out through the skin 102 with the driveline 100 and moved axially on the driveline cable 103 to position the sleeve 106 within the skin opening. If a velour-covered sleeve is used, the velour 110 may be engaged with the skin 102 as shown, or pushed further in so that the skin engages a smooth surface of the sleeve 106 and the velour 110 only contacts the subcutaneous tissue 101. The elastically loaded seals 115 and 116 of the inner bellows 107 and the outer bellows 108 are slid axially to adjust the initial bellows positions. Postoperative care and general cleaning and maintenance of the driveline exit site are unchanged from normal practice.

Replacement of the outer bellows 108 alone or the entire device assembly 105 may be accomplished in a clinical setting under sterile conditions. The old parts are removed while the driveline assembly 100 remains connected to the external power supply by cutting the parts longitudinally and slipping them off the driveline cable 103 from the side. Replacement parts are loaded in the proper order on a tool (not shown) that serves as an electrical extension for the driveline. The loaded extension tool is inserted between the driveline 100 and the power supply with only a brief power interruption, and the replacement parts are slid from the extension tool to the implanted driveline 100. The new parts are then assembled in place, and the extension tool is removed with a second brief power interruption. Preferably the extension tool presents a smooth exterior surface that covers the driveline connector and makes the sliding parts transfer easier and less likely to damage the stretched elastomeric components. The polymer split support ring 119 may added from the side. The device assembly 100 may also be retrofitted to the drivelines of compatible VAD systems implanted with a conventional driveline without a strain relief device to correct chronic interface injury problems using the device repair techniques described above. The preceding figures and descriptions show a preferred embodiment of the invention, but a number of variations are within its scope. A range of bellows configurations or other compliant seals known in the art are applicable. Further, while the double bellows arrangement shown provides redundancy and excludes both subcutaneous fluids and cells and external contaminants from the annular volume between the device assembly 105 and the cable 103, the device will function with only one compliant seal. If experience shows that outside bellows damage and replacement are rare events, the sleeve and two bellows might be combined into a single component, eliminating the bellows-sleeve connection and the split support ring. Optionally, the annular volume 120 between the device assembly 105 and of the cable 103 might be filled with a biocompatible gel that provides functions such as lubrication and antibacterial action.

What is claimed is:

1. A strain relief device comprising a sleeve surrounding a tube-like connecting member passing through a patient's skin that connects a medical device implanted within the patient to support apparatus outside the patient wherein the sleeve forms an adherent interface with the skin, the connecting member is free to move axially and rotationally within the sleeve, the device further comprises at least one compliant seal between the sleeve and the connecting member allowing relative motion between the tube-like connecting member and the sleeve, thereby reducing stress on the adherent interface between the skin and the sleeve while separating the patient's internal tissue from the external environment.

2. The strain relief device of claim 1 wherein the compliant seal or seals comprise bellows compliant in the axial direction, rotary direction, or both.

3. The strain relief device of claim 2 wherein the compliant bellows are formed of elastomer and are fixed to the sleeve and form elastically-loaded seals with the tube-like connecting member, wherein the elastically-loaded seals may be repositioned on the tube-like connecting member by sliding.

4. The strain relief device of claim 1 wherein the tube-like connecting member incorporates an enlarged outer end, and the sleeve and compliant seal or seals have sufficiently elasticity that they may be passed over the enlarged outer end without damage.

5. The strain relief device of claim 1 wherein the tube-like connecting member is the driveline of an implantable ventricular assist device connecting the implanted pump to the external power supply and control system.

6. The strain relief device of claim 1 wherein at least a portion of the sleeve outside diameter is covered by velour.

7. The strain relief device of claim 1 wherein a first compliant seal is positioned to seal between the sleeve and the tube-like connecting member inside the patient's body and a second compliant seal is positioned to seal between the sleeve and the tube-like connecting member outside the patient's body.

8. The strain relief device of claim 7 wherein the second compliant seal located outside the patient's body may be separated from the sleeve and replaced.

9. The strain relief device of claim 7 wherein the annular volume between the sleeve and seal assembly and the tube-like connecting member is filled with a liquid or gel having lubricating and/or medicinal properties.

10. The strain relief device of claim 8 further comprising a split polymer support ring that may be flexed open such that it may be inserted or removed from the tube-like connecting member from the side.

11. A method of reducing the stresses applied to an adherent interface between a patient's skin and an emerging portion of an implanted object comprising:
interposing a sleeve between the implanted object and the opening in the patient's skin such that the skin adheres to the sleeve rather than the implanted object; and
providing one or more compliant seals between the sleeve and the implanted object that mechanically isolate motions of the implanted object from the sleeve and its adherent interface with the skin while providing a barrier between the patient's internal tissue and the outside environment.

12. The method of claim 11 wherein the sleeve and seals are assembled on the implanted object prior to the implantation procedure and positioned in the skin interface as part of the procedure.

13. A method of installing all or a portion of a strain relief device comprising a sleeve and compliant seals surrounding a tube-like connecting member passing through a patient's skin connecting a previously implanted medical device and an external support device comprising:
removing any strain relief device components to be replaced;
loading the new elastomeric components on an tool that serves as a functional extension of the tube-like connecting member;
disconnecting the tube-like connecting member from the external support device and immediately connecting the loaded extension tool between the external support device and the implanted tube to restore system function;
sliding the new components from the extension tool to the tube-like connecting member utilizing the component elasticity to pass over the connector;
adding any components such as split polymer support rings to the tube-like connecting member from the side;
assembling the strain relief device on the tube-like connecting member, and adjusting its position relative to the skin interface;
removing the extension tool and immediately reconnecting the tube-like connecting member to the external support device to restore system function;
thereby carrying out the strain relief device installation with minimal interruption of the system function.
